(19) United States
(12) Patent Application Publication
(10) Pub. No.: US 2023/0116621 A1
(43) Pub. Date: Apr. 13, 2023

(54) DRUG DELIVERY CARRIER INCLUDING PLGA AND BETA-CYCLODEXTRIN CONTAINING DRUG

(71) Applicants: SELJIN CO.LTD., Seongnam-si (KR); SUNGKWANG MEDICAL FOUNDATION, Seoul (KR)

(72) Inventors: Seil SOHN, Seoul (KR); Wan Kyu KO, Suwon-si (KR); Daye LEE, Seongnam-si (KR); Seong Jun KIM, Gwangju-si (KR); Gong Ho HAN, Seoul (KR)

(73) Assignees: SELJIN CO.LTD., Seongnam-si (KR); SUNGKWANG MEDICAL FOUNDATION, Seoul (KR)

(21) Appl. No.: 17/502,181

(22) Filed: Oct. 15, 2021

(30) Foreign Application Priority Data
Oct. 7, 2021 (KR) 10-2021-0133361

(51) Int. Cl. A61K 47/40; A61K 47/34; A61K 9/14

(52) U.S. Cl. CPC A61K 47/40; A61K 47/34; A61K 9/146

(57) ABSTRACT

Provided is a drug delivery carrier including PLGA and β-cyclodextrin containing a drug. According to the drug delivery carrier, the time during which a drug stays in the living body may be prolonged, and due to the biodegradation thereof, few side effects occur.

### CROSS-REFERENCE TO RELATED APPLICATION

This application is based on and claims priority under 35 U.S.C. § 119 to Korean Patent Application No. 10-2021-0133361, filed on Oct. 7, 2021, in the Korean Intellectual Property Office, the disclosure of which is incorporated by reference herein in its entirety.

### FIELD OF THE INVENTION

The present invention relates to a drug delivery carrier including PLGA and a 3-cyclodextrin containing a drug.

### BACKGROUND OF THE INVENTION

In the case where pain receptor nerve ending is exposed to a mechanical, thermal, chemical or other harmful stimulus, pain may occur, in general. These pain receptors may transmit signals to the central nervous carrier, and then to the brain, along the centripetal nerve cells. When a human feels pain, one or more problems associated with these senses are involved, a decrease in function, a decrease in mobility, a complexity of sleep patterns, and a poor quality of life may occur, although not limited thereto.

Pain causes include, but are not limited to, damage that may be derived from inflammation, wounds, diseases, muscle stress, neuropathic cases or syndrome outbreaks, and surgical or hazardous physical, chemical or thermal accidents, or biological agent infections.

In order to treat pain, a non-surgical procedure, in which a drug is directly administered to a pain-causing area, is mainly used. However, since the drug in such a solution state is released instantly immediately after being administered, the effect of the drug does not last long. Therefore, pain symptoms occur again in the patient several days after the administration. In addition, frequently, the topical anesthetics used together unintentionally flow into the motor nerves, causing unwanted paralysis of upper and lower limbs.

Therefore, there is a need for a drug delivery carrier to solve these problems.

### SUMMARY OF THE INVENTION

One or more embodiments include a drug delivery carrier including: polylactic-co-glycolic acid (PLGA); and β-cyclodextrin containing a drug, wherein PLGA and the β-cyclodextrin are linked to each other by a linker.

One or more embodiments include a method of delivering a drug into a subject in need thereof, the method including administering, to the subject, a drug delivery carrier including PLGA and β-cyclodextrin containing a drug, wherein PLGA and the β-cyclodextrin are linked to each other by a linker.

One or more embodiments include a pharmaceutical composition for preventing or treating a pain disorder, the pharmaceutical composition including PLGA and β-cyclodextrin containing a pain treatment agent, wherein PLGA and the β-cyclodextrin are linked to each other by a linker.

One or more embodiments include a method of preventing or treating a pain disorder, the method including administering, to a subject in need thereof, a composition including: PLGA and β-cyclodextrin containing a pain treatment agent, wherein PLGA and the β-cyclodextrin are linked to each other by a linker.

One or more embodiments include a method of preparing the drug delivery carrier.

Additional aspects will be set forth in part in the description which follows and, in part, will be apparent from the description, or may be learned by practice of the presented embodiments of the disclosure.

An aspect of the present disclosure provides a drug delivery carrier including PLGA and β-cyclodextrin containing a drug, wherein PLGA and the β-cyclodextrin are linked to each other by a linker.

Another aspect of the present disclosure provides a method of delivering a drug into a subject in need thereof, the method including administering, to the subject, a drug delivery carrier including PLGA and β-cyclodextrin containing a drug, wherein PLGA and the β-cyclodextrin are linked to each other by a linker.

The term “PLGA” used herein refers to a polylactide-co-glycolic acid and is a polymer in which monomers of glycolic acid and lactic acid are cross-linked with each other. PLGA may be a block copolymer or a random copolymer. PLGA is a biodegradable material that may be decomposed by bodily fluids in vivo or microorganisms.

In an embodiment, PLGA and the β-cyclodextrin may each have a thiol group. Accordingly, the thiol group of PLGA and the thiol group of the β-cyclodextrin may be connected to each other by a disulfide bond. In an embodiment, the linker may be a disulfide bond, and the thiol group of PLGA and the thiol group of the β-cyclodextrin may form a disulfide bond.

PLGA may have a glycolic acid/lactic acid ratio of about 5:1 to about 1:5, for example, about 4:1 to about 1:4, about 3:1 to about 1:3, or about 2:1 to about 1:2. In an embodiment, the ratio of a glycolic acid to a lactic acid in PLGA may be 3:1. The drug delivery carrier may have a porous structure. The porosity of the porous structure may be represented by a volume percentage (vol %), and the porosity of the drug delivery carrier may be from about 30 vol % to about 50 vol %. Here, the porosity may refer to a ratio of the total volume of the pores to the total volume of the drug delivery carrier.

In addition, in the porous structure, the pore average diameter may be from about 500 nm to about 5 μm, from about 500 nm to about 3 μm, or from about 1 μm to about 2 μm.

The types of pharmaceutically active ingredients that can be delivered into a subject using a drug delivery carrier include pain treatment agents, anticancer agents, contrast agents (dyes), hormones, antihormones, vitamins, calcium agents, inorganic agents, saccharides, organic acid agents, protein amino acid agents, antidotes, enzyme agents, metabolic agents, diabetes combination agents, tissue revitalization agents, chlorophyll agents, pigment agents, tumor medications, tumor treatment agents, radiopharmaceutical products, tissue cell diagnostic agents, tissue cell treatment agents, antibiotic agents, antiviral agents, combined antibiotics, chemotherapeutic agents, vaccines, toxins, toxoids, antitoxins, leptospirin serum, blood preparations, biological preparations, analgesics, immunogenic molecules, antihistamines, allergy medications, non-specific immunogen preparations, anesthetic agents, stimulants, neuropsychiatric solvents, small molecule compounds, nucleic acids, aptamers, antisense nucleic acids, oligonucleotides, peptides, siRNAs, and microRNAs.

In an embodiment, the drug may be a pain treatment agent or an anesthetic agent.

The pain treatment agent may be a steroidal anti-inflammatory drug or a non-steroidal anti-inflammatory drug (NSAIDs).

The pain treatment agent may include at least one selected from celecoxib, diclofenac, diflunisal, piroxicam, meloxicam, etodolac, mefenamic acid, meclofenamic acid, ibuprofen, indometacin, ketoprofen, ketorolac, nabumetone, naproxen, nimesulide, sulindac, tepoxalin, tolmetin, neostigmine, magnesium, atropine, dexamethasone, prednisolone, prednisone, methyl prednisolone, triamcinolone, hydrocortisone, deflazacourt, betamethasone, budenoside, ketorolac, octreotide, ziconitide, droperidol, methotrexate, and haloperidol.

The anesthetic agent may be a topical anesthetic agent.

The anesthetic agent may be at least one selected from bupivacaine, levobupivacaine, ropivacaine, prilocaine, mepivacaine, benzocaine, tetracaine, and lidocaine.

In an embodiment, in the drug delivery carrier, a drug may be released in a sustained manner.

The terms “sustained release” and “sustained release manner” used herein include various terms that may be used interchangeably when pharmaceutical descriptions are provided, such as extended release, delayed release, sustained release, controlled release, sustained action release, specific release, and target release, and may indicate that one or more treatment agent(s) administered into a human or other mammalian body continuously or continuously release one or more treatment agents for a certain period of time to retain a therapeutic level sufficient to achieve a desired therapeutic effect for a certain period of time. Continuous or sustained release includes release resulting from biodegradation of the in vivo drugs, nanofibers or matrix or components thereof, or from metabolic modification or dissolution of the treatment agent(s) or conjugate of the treatment agent(s).

In an embodiment, the drug delivery carrier may be produced by linking a drug-loaded β-cyclodextrin to a PLGA. Specifically, instead of linking β-cyclodextrin to PLGA and then loading the drug, the drug may be loaded onto β-cyclodextrin, which is then linked to PLGA.

In addition, in an embodiment, PLGA may be subjected to electrospinning to form nanofibers after the thiol end group is formed. Specifically, instead of forming a thiol end group at PLGA and then nanofibers are formed by electrospinning, only after a thiol end group is formed at PLGA, nanofibers are formed by electrospinning.

The drug delivery carrier according to an embodiment prepared as described above may retain the porous structure well without any deformation in the structure of PLGA.

Another aspect provides a pharmaceutical composition for preventing or treating a pain disorder, the pharmaceutical composition including PLGA and β-cyclodextrin containing a pain treatment agent, wherein PLGA and the β-cyclodextrin are linked to each other by a linker.

Another aspect of the present disclosure provides a method of preventing or treating a pain disorder, the method including administering, to a subject in need thereof, a composition including PLGA and β-cyclodextrin containing a pain treatment agent, wherein PLGA and the β-cyclodextrin are linked to each other by a linker.

According to another aspect of the present disclosure, there is provided a use of the composition including PLGA and β-cyclodextrin containing a pain treatment agent, wherein PLGA and the β-cyclodextrin are linked to each other by a linker, for use in the prevention or treatment of a pain disorder.

In an embodiment, PLGA and the β-cyclodextrin may each have a thiol group. Accordingly, the thiol group of PLGA and the thiol group of the β-cyclodextrin may be connected to each other by a disulfide bond. In an embodiment, the linker may be a disulfide bond, and the thiol group of PLGA and the thiol group of the β-cyclodextrin may form a disulfide bond.

In an embodiment, the composition may further include an anesthetic agent.

In an embodiment, the pain disorder may be caused by any one selected from neuropathic pain, osteoarthritis, rheumatoid arthritis, fibromyalgia, back and musculoskeletal pain, spondylitis, intervertebral disk escape, spinal canal stenosis, juvenile rheumatoid arthritis, diabetic neuropathy, spontaneous pain, hypersensitivity pain, phantom limb pain, complex regional pain syndrome migraine, toothache, abdominal pain, ischemic pain, and post-operative pain.

For example, the composition may be used to treat one or more target tissue sites associated with: rheumatoid arthritis, osteoarthritis, sciatica, wrist tunnel syndrome, lower abdomen pain, leg pain, arm pain, cancer or tissue pain; and pain associated with injury or recovery of neck, chest and/or lumbar vertebrae or intervertebral disks, rotator cuff, joint, TMJ, tendon, ligament, muscle surgical wound sites or incision sites.

The terms “subject”, “individual” and “patient” herein are used interchangeably herein to refer to vertebrates, or mammals, or humans. Mammals include, but are not limited to, murine, monkey, human, farm animal, sports animal and pet. Mammals may include tissues, cells and progeny of biological entities obtained in vivo or cultured in vitro.

The term “treatment agent” or “pharmaceutical composition” as used herein refers to a molecule or compound that provides some beneficial effects when administered to a subject. The beneficial effects may include: enabling diagnostic decisions; amelioration of disease, symptoms, disability, or conditions; reduction or prevention of the onset of a disease, symptom, disability, or disorder; and responding to disease, symptoms, disability, or conditions.

The terms “treat” or “treatment” or “amelioration” or “improvement” used herein are used interchangeably. These terms refer to methods of obtaining favorable or desired results, including, but not limited to, therapeutic benefits and/or prophylactic benefits. Therapeutic benefits refer to any therapeutically significant improvement in or effect on one or more diseases, disorders or symptoms under treatment. For prophylactic benefits, the composition may be administered to a subject at risk of developing a particular disease, disorder or symptom, or to a subject reporting one or more physiological symptoms of a disease, even though the disease, disorder or symptom has not yet been present.

The term “effective amount” or “therapeutically effective amount” used herein refers to an amount of an agent sufficient to cause favorable or desired results. The therapeutically effective amount may vary depending on one or more selected from a subject to be treated, a condition to be treated, the weight and age of the subject, the severity of the condition, the mode of administration, and the like. Those skilled in the art may easily determine the therapeutically effective amount. Furthermore, the term corresponds to a capacity that will provide an image for detection by any of the imaging methods described herein. The particular dose may vary depending on one or more selected from a particular agent selected, the following administration method, co-administration of the same with another compound, the timing of administration, the tissue being imaged, and the body delivery carrier carrying the same.

The pharmaceutical composition may be administered parenterally at the time of clinical administration and may be used in the form of general pharmaceutical preparations. Parenteral administration may refer to administration via a route of administration other than oral route, such as rectum, vein, peritoneal, muscle, artery, transdermal, nasal, inhalation, eyeball, or subcutaneous route. When the pharmaceutical composition of the present disclosure is used as a medicine, one or more active ingredients exhibiting the same or similar functions may be additionally included.

In addition, the pharmaceutical composition may be delivered topically, and this delivery may include delivery of one or more drugs to be placed in the inside a tissue, for example, a nerve system nerve root or brain region or an adjacent region thereto (e.g., within about 0.1 cm or within about 10 cm). For example, a topically delivered drug dose may be, for example, within 10%, 20%, 30%, 40%, 50%, 60%, 70%, 80%, 90%, 95%, 99%, or 99.9% of the oral or injectable dose. Thus, systemic side effects such as liver aminotransferase elevation, hepatitis, liver failure, myopathy, constipation, etc., are reduced or eliminated.

Those skilled in the art may understand that the composition may be administered to a target site using a drug delivery device such as a syringe, a gun drug delivery device, or a “cannula” or “needle” which may be part of any suitable device for the application of a drug to the target organ or anatomical region. The cannula or needle is designed to cause minimal physical and physiological trauma to the patient.

The cannula or needle may include, for example, a tube made from a material including polyurethane, polyurea, polyether (amide), PEBA, thermoplastic elastic olefin, polyester, styrene thermoplastic elastomer, steel, aluminum, stainless steel, titanium, metal alloys having high nonferrous metal content and relatively low iron ratios, carbon fibers, glass fibers, plastics, ceramics, or combinations thereof.

The cannula or needle may optionally include one or more inclined zones. In one or more embodiments, the cannula or needle may be sloped. In addition, the cannula or needle may have a tip shape required for accurate treatment of the patient depending on the implantation site. Examples of tip shapes include Trepin, Kurnande, Beres, Huber, Seldingger, Ziva, Francin, Vias, Crawford, deflection tips, Hustide, Lancet, and Tui. In one or more embodiments, the cannula or needle may be non-lumen treated and have a cover covering the same to avoid unwanted needle pricking.

Among other things, the dimensions of the hollow cannula or needle may vary depending on the implantation site. For example, the epidural space width may be as small as about 3 mm to about 5 mm in the chest region and about 5 mm to about 7 mm in the waist region. Thus, in one or more embodiments, a needle or cannula may be designed for this particular area. In one or more embodiments, the cannula or needle may be inserted along the inflammatory nerve root by, for example, applying a hole-through approach in the spinal hole space, and the composition may be implanted in the target site for the treatment of conditions. Typically, the hole-through approach includes passing through a hole in the vertebrae to have an access to the space in the vertebrae.

Some examples of the length of cannula or needle include, but are not limited to, about 50 mm to about 150 mm, for example, about 65 mm for epidural pediatric, about 85 mm for standard adult, and about 110 mm for obese adult patients. The thickness of the cannula or needle may also vary depending on the implantation site. In one or more embodiments, the thickness thereof may include, but is not limited to, the range from about 0.05 mm to about 1.655 mm. The gage of the cannula or needle may be the widest or narrowest diameter or a diameter therebetween, for insertion into the human or animal body. The widest diameter thereof may generally be about 14 gage and the narrowest diameter thereof may be about 22 gage. In one or more embodiments, the gage of the needle or cannula may be from about 18 gage to about 22 gage.

In one or more embodiments, the cannula or needle may include a radiographic sign indicating a point at or around a lower part of the skin so that the user may apply any diagnostic imaging procedures to accurately position a site or a surrounding depot location. The diagnostic imaging procedure includes, for example, an X-ray image or fluoroscopy. Examples of such radiolabels include, but are not limited to, barium, bismuth, tantalum, tungsten, iodine, calcium and/or metallic beads or particles.

In one or more embodiments, the needle or cannula may be visualized with ultrasound, fluoroscopy, X-ray, or other imaging techniques by including a transparent or translucent portion. In such an example, the transparent or translucent portion may include a radiopaque material or ultrasonic reflective topography to increase a needle or cannula contrast compared to a point where such material or topography does not exist.

In one or more embodiments, when the target site is a spinal region, a portion of the bodily fluid (e.g., spinal fluid) is first sucked through a cannula or needle and then the composition is administered (e.g., placed, dipped, infused, or implanted, etc.). The target site may be re-hydrated (e.g., bodily fluid replenished again) and due to this water-soluble environment, the drug is released from the composition.

In addition, the composition may be delivered to any site under the skin, for example, to at least one muscle, ligament, tendon, cartilage, spinal disk, intervertebral foramen space, the surrounding of the spinal nerve root, or the spinal canal. However, embodiments of the present disclosure are not limited thereto.

Parenteral administration includes, for example, infusion pumps using a catheter through which a pharmaceutical composition (e.g., a combination of a sedative and an anti-inflammatory drug) is administered around the spine or one or more inflammatory joints, implantable mini-pumps that may be inserted at or around a target site, and implantable controlled release devices or sustained release delivery carriers that may release a certain amount or an intermittent amount of statin per hour.

When the pharmaceutical composition is formulated, diluents or excipients of the related art, such as fillers, extenders, binders, wetting agents, disintegrants, and surfactants, may be used. Formulations for parenteral administration include sterile aqueous solutions, non-aqueous solvents, suspensions, emulsions, lyophilized formulations, and suppositories. As the non-aqueous solvent and the suspension solvent, propylene glycol, polyethylene glycol, vegetable oil such as olive oil, injectable ester such as ethyl oleate, and the like may be used. Witepsol, Macrogol, Tween 61, cacao butter, Liulin paper, glycerogelatin, and the like may be used as a base of the suppository.

In addition, the pharmaceutical composition may be mixed with various carriers which are allowed as a drug, such as physiological saline or an organic solvent. To increase stability or absorbability, carbohydrates such as glucose, sucrose or dextran, antioxidants such as ascorbic acid or glutathione, chelating agents, low molecular protein or other stabilizers may be used as a drug.

The effective dose of the pharmaceutical composition may be about 0.01 mg/kg to about 100 mg/kg, for example, about 0.1 mg/kg to about 10 mg/kg, and may be administered once to three times a day.

Another aspect provides a drug delivery carrier preparation method including: forming a thiol end group in PLGA; entrapping a drug in β-cyclodextrin having a thiol group; and linking the PLGA and the β-cyclodextrin via a disulfide bond.

In an embodiment, the method may further include electrospinning PLGA having the thiol end group therein.

The drug delivery carrier according to an embodiment prepared by using the method may retain the porous structure well without any deformation in the structure of PLGA.

The terms, methods, effects, and the like described above are equally applied into each disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A, FIG. 1B, and FIG. 1C show diagrams illustrating a method of preparing a polylactic-co-glycolic acid (PLGA)-CD-DEX-RVC according to an embodiment of the present disclosure:

FIG. 1A shows a diagram illustrating a synthesis process and the formula of PLGA-SH; FIG. 1B shows a diagram illustrating the structure of SH-β-CD; and FIG. 1C shows a diagram illustrating the process of entrapping a drug in SH-β-CD and linking the resultant structure to electrospun PLGA-SH;

FIGS. 2A-2E show images of the surface and porosity of PLGA-CD-DEX-RVC according to an embodiment and other nanofibers:

FIG. 2A shows a scanning electron microscope (SEM) image of PLGA-SH; FIG. 2B shows a SEM image of a PLGA-CD-DEX-RVC according to an embodiment; FIG. 2C shows a SEM image of PLGA-CD-DEX-RVC prepared by entrapping a drug in PLGA-S-S-CD; FIG. 2D shows a graph in which the porosity of PLGA-CD-DEX-RVC according to an embodiment is compared with of the porosities of PLGA, PLGA-SH and PLGA-CD+DEX-RVC; and FIG. 2E shows a diagram illustrating formulae of dexamethasone and ropivacaine entrapped in PLGA-CD-DEX-RVC according to an embodiment;

FIG. 3A, FIG. 3B, and FIG. 3C show graphs in which an element contained in PLGA-CD-DEX-RVC is compared with PLGA, PLGA-SH, and PLGA-S-S-CD according to an embodiment of the present disclosure:

FIG. 3A shows a graph of measurements of elements of PLGA, PLGA-SH, PLGA-S-S-CD, and PLGA-CD-DEX-RVC in a wide range; FIG. 3B shows a graph of measurements only in a narrow range in which F 1s is detected; and FIG. 3C shows a graph of measurements only in a narrow range in which N 1s is detected;

FIG. 4A and FIG. 4B show images from which the cytotoxicity of PLGA-CD-DEX-RVC according to an embodiment was confirmed:

FIG. 4A shows an image of cells from which the cytotoxicity of PLGA and PLGA-CD-DEX-RVC (Scale bar: 200 μm) was confirmed; and FIG. 4B shows a diagram from which the cytotoxicity of PLGA and PLGA-CD-DEX-RVC according to an embodiment was confirmed;

FIG. 5A and FIG. 5B show a diagram of the drug release rate of PLGA-CD-DEX and PLGA-CD-RVC according to an embodiment:

FIG. 5A shows a graph of the drug release time of the group in which PLGA was simply loaded with dexamethasone (PLGA+DEX) and the group in which dexamethasone was entrapped in SH-β-CD and then PLGA was linked thereto (PLGA-CD-DEX); and FIG. 5B shows a graph of the drug release time of the group in which PLGA was simply loaded with ropivacaine (PLGA+RVC) and the group in which dexamethasone was entrapped in SH-β-CD and then PLGA was linked thereto (PLGA-CD-RVC);

FIG. 6A, FIG. 6B, and FIG. 6C show images from which the anti-inflammatory effect of PLGA-CD-DEX-RVC according to an embodiment was confirmed:

FIG. 6A shows images of immunohistofluorescence-stained cells from which the differentiation into M1/M2 macrophages was confirmed (Scale bar: 50 μm); FIG. 6B shows a graph of the quantified expression of the inflammatory factor iNOS; and FIG. 6C shows a graph of the quantified expression of the inflammatory factor CD206;

FIG. 7A and FIG. 7B show images from which the pain reduction effect of PLGA-CD-DEX-RVC according to an embodiment was confirmed:

FIG. 7A shows a schematic diagram showing an animal experiment using a chronic constriction injury (CCI) animal model and the effect of PLGA-CD-DEX-RVC; and FIG. 7B shows a graph showing the pain reduction effect of PLGA-CD-DEX-RVC obtained through pain evaluation (cold allodynia);

FIG. 8A and FIG. 8B show in vivo decomposition effects of PLGA-CD-DEX-RVC according to an embodiment:

FIG. 8A shows images showing the degradation of PLGA-CD-DEX-RVC in vivo over time; and FIG. 8B shows a graph showing the degradation of PLGA-CD-DEX-RVC in vivo over time;

FIGS. 9A-9E show the expression levels of TRPV1, a pain factor, when PLGA-CD-DEX-RVC according to an embodiment was used for the treatment after nerve injury, wherein the expression levels were identified by immunofluorescence staining:

FIG. 9A shows a diagram illustrating that a pain signal is transferred from sensory neurons of the dorsal root ganglia (DRG) to the dorsal horn of spinal cord; FIG. 9B shows an image showing the expression of TRPV1, a pain marker, in DRG (Scale bar: 20 μm); FIG. 9C shows a graph of the quantified expression of TRPV1, a pain marker, in DRG; FIG. 9D shows an image showing the expression of TRPV1, a pain marker, in the spinal cord of dorsal horn (Scale bar: 100 μm); and FIG. 9E shows a graph of the quantified expression of TRPV1, a pain marker, in the spinal cord of dorsal horn; and

FIGS. 10A-10D show the expression levels of Iba1, an inflammatory factor, when PLGA-CD-DEX-RVC according to an embodiment was used for treatment after nerve injury, wherein the expression levels were identified by immunofluorescence staining:

FIG. 10A shows an image showing the expression of Iba1, an inflammatory marker, in DRG (Scale bar: 20 μm); FIG. 10B shows a graph of the quantified expression of Iba1, an inflammatory marker, in DRG; FIG. 10C shows an image showing the expression of Iba1, an inflammatory marker, in the spinal cord of dorsal horn (Scale bar: 20 μm); and FIG. 10D shows a graph of the quantified expression of Iba1, an inflammatory marker, in the spinal cord of dorsal horn.

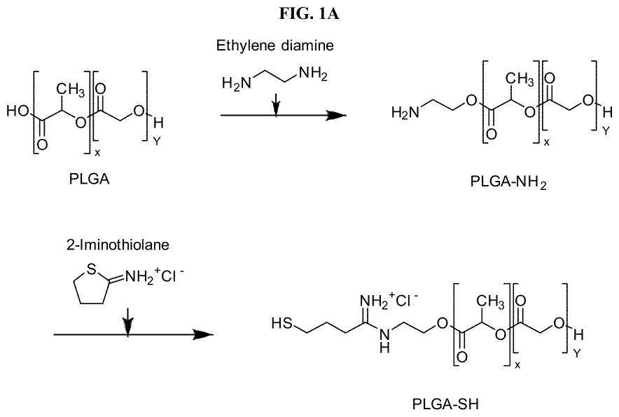

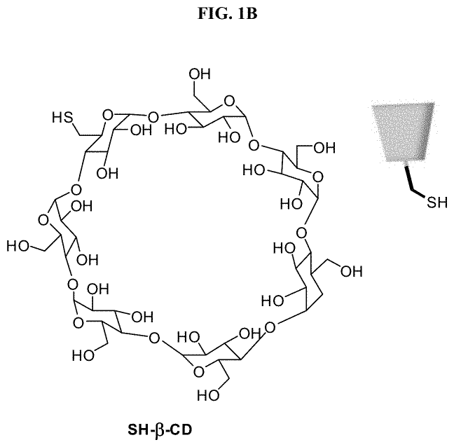

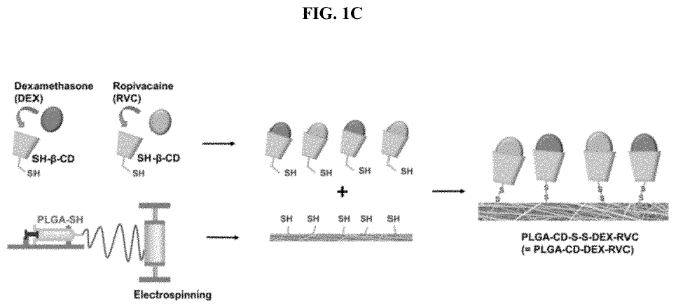

### DETAILED DESCRIPTION OF THE INVENTION

Hereinafter, the present disclosure will be described in more detail through Examples. However, these examples are intended to exemplarily describe the present disclosure, and the scope of the present disclosure is not limited to these examples.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

### Example 1. Preparation of Drug-Loaded PLGA-CD-DEX-RVC

Drug-loaded PLGA-CD was prepared as follows.

Specifically, polylactic-co-glycolic acid (PLGA) having a thiol (SH) end group was synthesized as follows. PLGA having a glycolic acid and lactic acid at a ratio of 75:25 was dissolved in dichloromethanol (DCM) together with N-hydroxysuccinimide and N,N′-dicyclohexylcarbodiimide at a molar ratio of 1:10:10, and then PLGA and ethylene diamine were added thereto at a molar ratio of 1:2. After 24 hours, after filtering using a 0.45 μm filter, the filtered solution was precipitated in cold diethyl ether and vacuum dried at room temperature to form an amine (NH₂) end group in PLGA. The synthesized PLGA-NH₂ was dissolved in DCM again, and a 5 M 2-iminothiolane hydrochloride methanol solution was added thereto to react for 1 day. The mixture was again precipitated in cold diethyl ether and vacuum dried to synthesize PLGA-SH having a thiol (SH) end group. The powder-type PLGA-SH synthesized as described above was dissolved at a concentration of 12 wt/v % in hexafluoroisopropanol (HFIP) and then electrospun at 200 rpm.

Next, the same molar concentration of dexamethasone (DEX) or ropivacaine (RVC) was added to a 1 μg/ml of mono-(6-mercapto-6-deoxy)-β-cyclodextrin (SH-β-CD) (purchased from AARON PHARMATEC.Ltd) solution, and each of these solutions was mixed by using a stirrer for 24 hours, and then freeze-dried to prepare SH-β-CD-DEX or SH-β-CD-RVC in a powder form in which DEX or RVC was entrapped.

Then, 5 μg/ml of (SH-β-CD-DEX+SH-β-CD-RVC/distilled water) in which a ratio of SH-β-CD-DEX to SH-β-CD-RVC was 1:1, was prepared, and then the resulting mixture was attached to the PLGA-SH nanofiber by disulfide bond (—S—S—), thereby preparing the final product, PLGA-CD-DEX-RVC nanofiber.

### Experimental Example 1. Identification of Surface and Porosity of PLGA-CD-DEX-RVC

The surface structure and porosity of the PLGA-CD-DEX-RVC nanofibers prepared in Example 1 were confirmed by SEM.

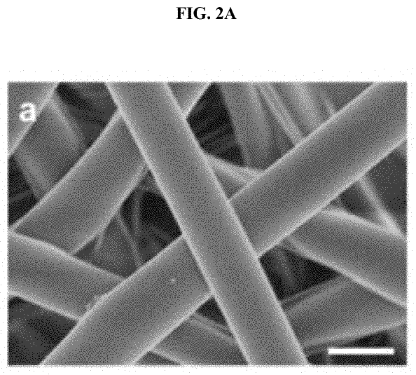

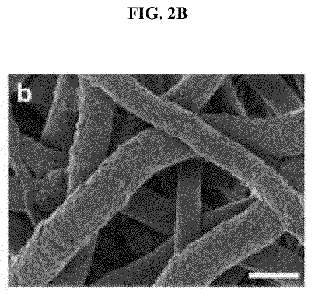

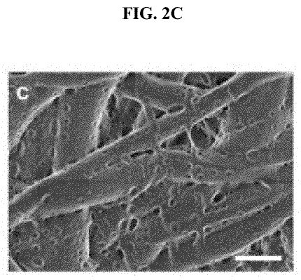

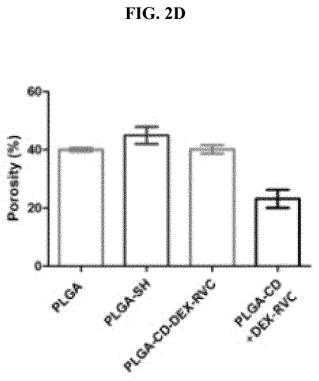

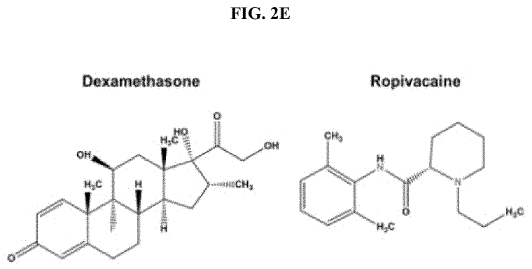

FIG. 2 shows images of the surface and porosity of PLGA-CD-DEX-RVC according to an embodiment and other nanofibers:

FIG. 2A shows a SEM image of PLGA-SH; FIG. 2B shows a SEM image of a PLGA-CD-DEX-RVC according to an embodiment; FIG. 2C shows a SEM image of PLGA-CD-DEX-RVC prepared by entrapping a drug in PLGA-S-S-CD; FIG. 2D shows a graph in which the porosity of PLGA-CD-DEX-RVC according to an embodiment is compared with of the porosities of PLGA and PLGA-SH; and FIG. 2E shows a diagram illustrating formulae of dexamethasone and ropivacaine entrapped in PLGA-CD-DEX-RVC according to an embodiment.

As a result, it was confirmed that even when compared with the case where the PLGA-SH solution was electrospun and the nanofibers were not bound with the drug (FIG. 2A), the PLGA-CD-DEX-RVC prepared in Example 1 also had a significantly structural shape without broken or pierced nanofibers as shown in FIG. 2B.

On the other hand, unlike the method of Example 1, in the case of the nanofiber PLGA-CD+DEX-RVC, which was prepared by obtaining PLGA-S-S-CD in which SH-β-CD was bound to PLGA-SH nanofibers, and then adding DEX or RVC drugs thereto, followed by vortexing, as shown in FIG. 2C, there were no nanofibers having normal shapes, that is, nanofibers had holes, were broken or melted.

In addition, porosity measurements show that, as shown in FIG. 2D, the degrees of porosity of PLGA nanofibers, PLGA-SH nanofibers, and PLGA-CD-DEX-RVC nanofibers prepared in Example 1 (the third graph of FIG. 2D) were almost similar to each other. On the other hand, as shown in FIG. 2C, it was confirmed that the degree of porosity of the nanofiber PLGA-CD+DEX-RVC, which was prepared by preparing PLGA-S-S-CD and then adding DEX or RVC drugs thereto, followed by vortexing, was greatly reduced. Numerical data obtained by quantitatively measuring the degree of porosity are shown in Table 1. These results indicate that the PLGA-CD-DEX-RVC nanofibers prepared in Example 1 are suitable for use as a scaffold.

TABLE 1

| Group | Porosity (%) |
|---|---|
| PLGA | 40.02% ± 0.82 |
| PLGA-SH | 44.92% ± 4.10 |
| PLGA-CD-DEX-RVC | 40.11% ± 2.05 |
| PLGA-CD + DEX-RVC | 23.18% ± 4.37 |

### Experimental Example 2. Confirmation of Presence of Drug Bound to PLGA-CD-DEX-RVC

Whether DEX and RVC drugs were well attached onto the surface of the PLGA-CD-DEX-RVC nanofiber prepared in Example 1, was confirmed by X-ray photoelectron spectroscopy (XPS).

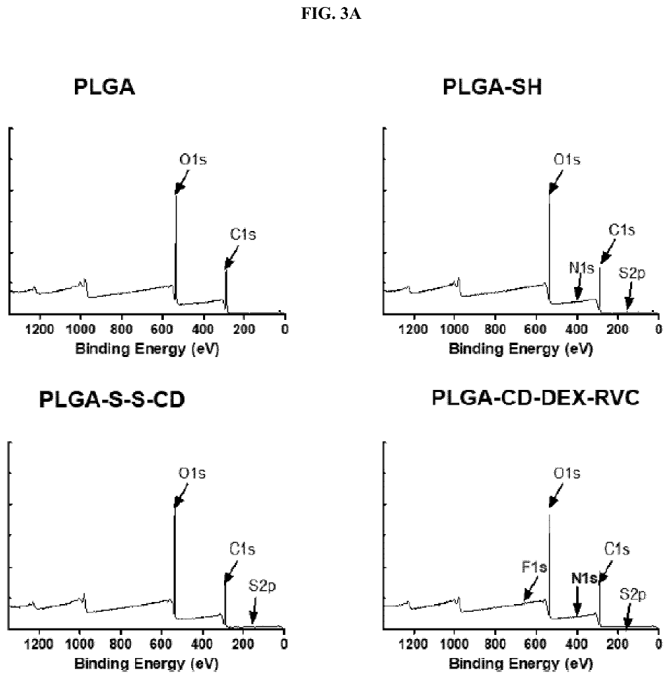

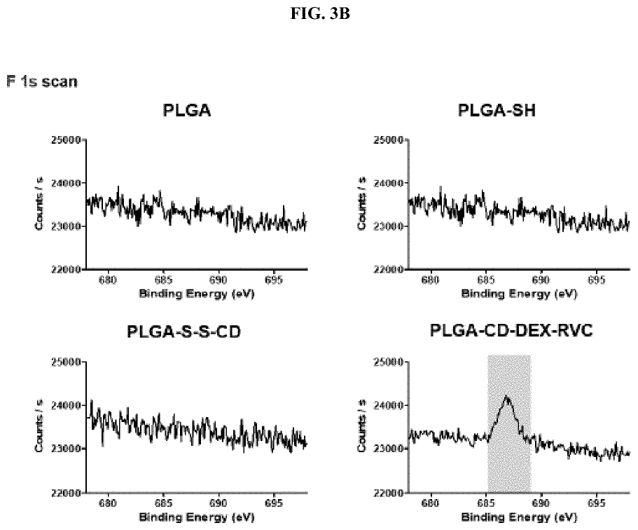

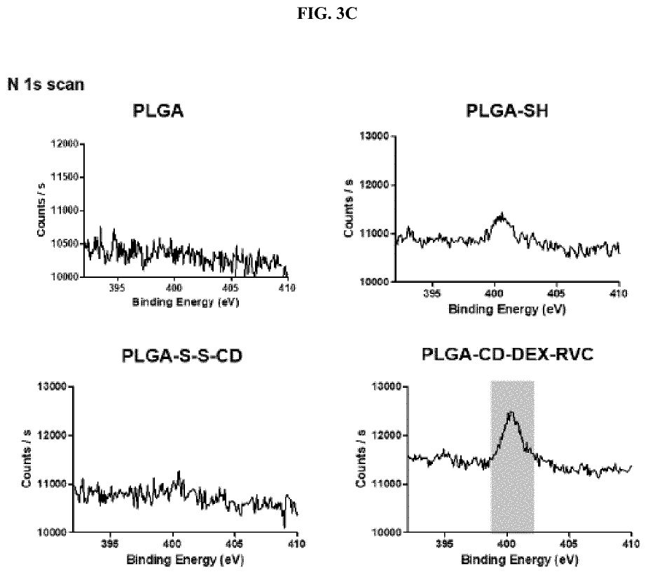

FIG. 3 shows a graph in which an element contained in PLGA-CD-DEX-RVC is compared with PLGA, PLGA-SH, and PLGA-S-S-CD according to an embodiment of the present disclosure:

FIG. 3A shows a graph of measurements of elements of PLGA, PLGA-SH, PLGA-S-S-CD, and PLGA-CD-DEX-RVC in a wide range; FIG. 3B shows a graph of measurements only in a narrow range in which F 1s is detected; and FIG. 3C shows a graph of measurements only in a narrow range in which N 1s is detected.

As shown in FIG. 3, it was confirmed that the F element present only in DEX and the N element present only in RVC were detected in PLGA-CD-DEX-RVC, and the F element and the N element were not detected in PLGA, PLGA-SH, and PLGA-S-S-CD to which the drugs were not attached.

These results indicate that the drugs are well attached onto the PLGA-CD-DEX-RVC nanofibers prepared in Example 1.

### Experimental Example 3. Cytotoxicity Confirmation

The cytotoxicity of the PLGA-CD-DEX-RVC nanofibers prepared in Example 1 was confirmed.

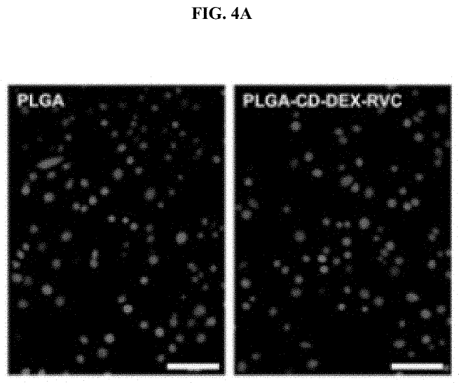

Specifically, to obtain bone marrow-derived macrophage (BMM) for confirming cytotoxicity, SD rats, an experimental animal, were sacrificed and bone marrow was extracted from femur and tibia, and BMM was separated according to the manual. The isolated BMM cells were cultured on PLGA or PLGA-CD-DEX-RVC nanofibers, and one day later, live and dead staining was performed to identify live cells and dead cells (FIG. 4A). Also, cytotoxicity was quantitatively confirmed by using a cytotoxicity assay kit (EZ-Cytox, Daeil Labservice, Korea) (FIG. 4B).

FIG. 4 shows images from which the cytotoxicity of PLGA-CD-DEX-RVC according to an embodiment was confirmed:

FIG. 4A shows an image of cells from which the cytotoxicity of PLGA and PLGA-CD-DEX-RVC (Scale bar: 200 μm) was confirmed; and FIG. 4B shows a diagram from which the cytotoxicity of PLGA and PLGA-CD-DEX-RVC according to an embodiment was confirmed.

As a result, as shown in FIGS. 4A and 4B, it was confirmed that compared to PLGA, PLGA-CD-DEX-RVC had similar cell viability and no cytotoxicity.

### Experimental Example 4. Confirmation of Drug Release Time

The drug release time of the PLGA-CD-DEX-RVC nanofibers prepared in Example 1 was confirmed.

Specifically, the drug release time of a group (PLGA-DEX or PLGA-DEX) in which the drug was simply loaded on PLGA without p-CD was compared with the drug release time of a group (PLGA-CD-DEX or PLGA-CD-RVC) in which each drug was entrapped in β-CD and then bound to PLGA. To determine the degree of release of the drug over time, each group was placed in DPBS and reacted in a shaker at 37° C. at 100 RPM. At 1, 4, 8, 12, 24 and 48 hours, the amount of drug released in the supernatant was quantified using UV-Vis spectrophotometer.

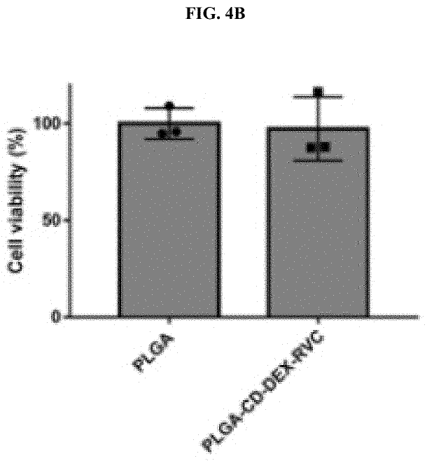

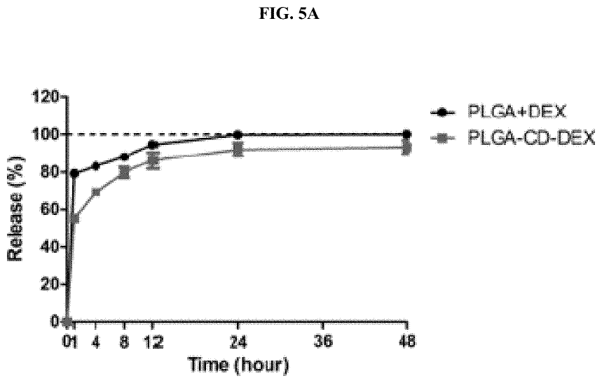

FIG. 5 shows a diagram of the drug release rate of PLGA-CD-DEX and PLGA-CD-RVC according to an embodiment:

FIG. 5A shows a graph of the drug release time of the group in which PLGA was simply loaded with dexamethasone (PLGA+DEX) and the group in which dexamethasone was entrapped in SH-β-CD and then PLGA was linked thereto (PLGA-CD-DEX); and FIG. 5B shows a graph of the drug release time of the group in which PLGA was simply loaded with ropivacaine (PLGA+RVC) and the group in which dexamethasone was entrapped in SH-β-CD and then PLGA was linked thereto (PLGA-CD-RVC).

As a result, as shown in FIGS. 5A and 5B, it was confirmed that in the case of the group in which the drug was simply loaded on PLGA, 100% of the drug was released before 24 hours, and in the case of the group in which the drug was entrapped in SH-β-CD and bound to PLGA, the drug was continuously released for more than 48 hours. These results indicate that nanofibers in which the drug was entrapped in SH-β-CD and then bound to PLGA can be used as a sustained-release drug delivery carrier that slowly releases the drug.

### Experimental Example 5. Confirmation of Anti-Inflammatory Effect

The anti-inflammatory effect of the PLGA-CD-DEX-RVC nanofibers prepared in Example 1 was confirmed in vitro by immunohistochemical staining.

Specifically, a total of five groups were used to perform cell experiments: 1) a control group not treated with LPS, 2) a group induced to differentiate into macrophages by treatment with 1 μg/ml of LPS; 3) a LPS+DEX/RVC group treated with 1 μg/ml of LPS, and 0.05 mg/mL of DEX and 0.05 mg/mL of RVC, 4) a LPS+PLGA+DEX/RVC group treated with 1 μg/ml of LPS and PLGA loaded with 0.05 mg/mL of DEX and 0.05 mg/mL of RVC, and 5) a LPS+PLGA-CD-DEX-RVC group treated with 1 μg/ml of LPS and PLGA-CD-DEX-RVC in which β-CD entrapping 0.15 mg of DEX and 0.15 mg of RVC was bound to PLGA.

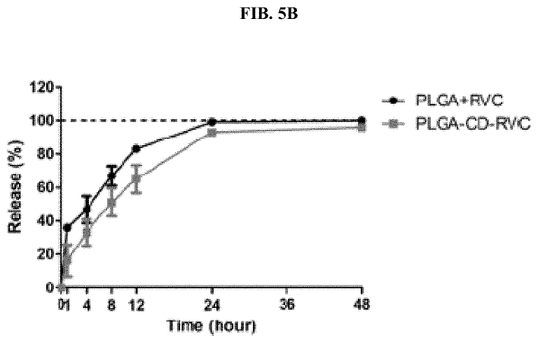

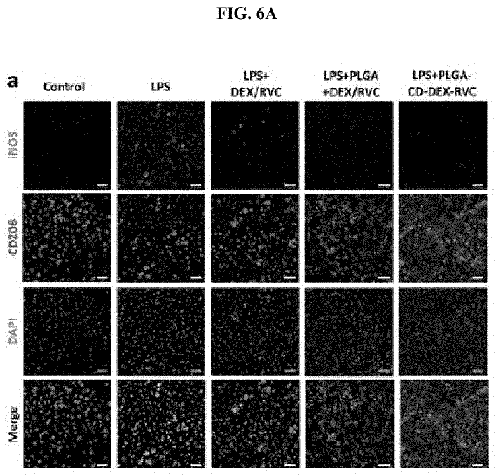

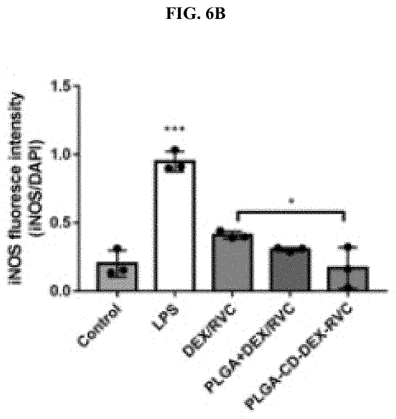

For each group, BMM cells (1.2×10⁵/well) were inoculated into 48-well culture plate and cultured. In the case of the groups 4) and 5), nanofibers were initially laid on the floor and inoculated with BMM cells. Then, the cells were treated with 1 μg/ml of LPS to induce an inflammatory reaction, and after 24 hours, the cells were immobilized by using 4% paraformaldehyde (PFA). When inflammation was induced in macrophages, an antibody against iNOS, which is a representative M1 marker secreted from macrophages, and an antibody against CD206 (Cluster of Differentiation 206), which is an M2 marker secreted to inhibit inflammation in macrophages, were used to perform staining using immunohistofluorescence, and expression levels were confirmed in a qualitative manner (FIG. 6A) and quantitative manner (FIGS. 6B and 6C). Rabbit anti-iNOS (1:500) and mouse anti-CD206 (1:500) were used as the primary antibodies, and donkey anti rabbit 647 (1:1000) and goat anti mouse 488 (1:1000) were used as the secondary antibodies, and after staining, the cells were mounted and images thereof were obtained by confocal microscopy.

FIG. 6 shows images from which the anti-inflammatory effect of PLGA-CD-DEX-RVC according to an embodiment was confirmed:

FIG. 6A shows images of immunohistofluorescence-stained cells from which the differentiation into M1/M2 macrophages was confirmed (Scale bar: 50 μm); FIG. 6B shows a graph of the quantified expression of the inflammatory factor iNOS; and FIG. 6C shows a graph of the quantified expression of the inflammatory factor CD206.

As a result, as shown in FIG. 6, it was confirmed that the group 5) treated with PLGA-CD-DEX-RVC showed a lower level of iNOS and a higher level of CD206 than the group 3) simply treated with DEX/RVC and the group 4) simply treated with PLGA loaded with DEX/RVC. These results indicate that PLGA-CD-DEX-RVC effectively inhibits inflammation compared to the treatment with the drug or the treatment with PLGA loaded with the drug.

### Experimental Example 6. Confirm the Effect of Reducing Neurogenic Pain

The pain reduction effect of the PLGA-CD-DEX-RVC nanofibers prepared in Example 1 was confirmed in vivo.

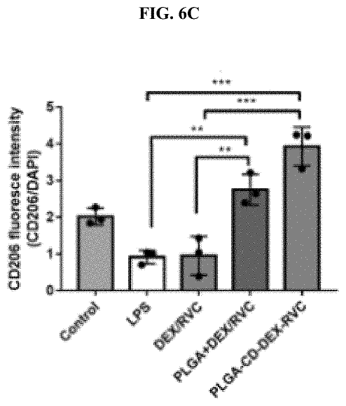

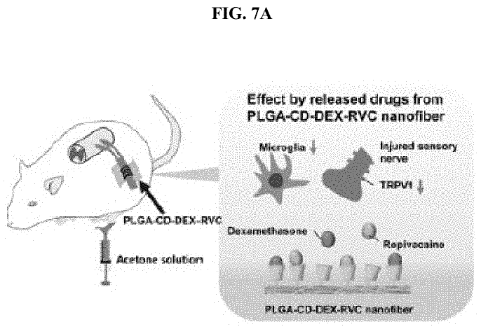

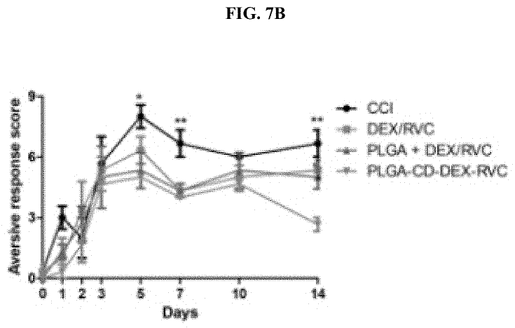

In detail, the sciatic nerve of a 8-week-old female SD rat was bound four times by using 4-0 nylon suture at 1 mm intervals to produce a chronic constriction injury (CCI) animal model (FIG. 7A). A total of four animal model groups were used in vivo, and the groups all received CCI damage: 1) a CCI group; 2) a DEX/RVC group in which 0.25 mg/kg of DEX and 0.25 mg/kg of RVC were injected in a solution state into the injured area, 3) a PLGA+DEX/RVC group in which the injured area was treated with PLGA nanofibers, simply loaded with 0.25 mg/kg of DEX and 0.25 mg/kg of RVC, and 4) a PLGA-CD-DEX-RVC group in which PLGA-CD entrapped 0.15 mg of DEX and 0.15 mg of RVC. Pain assessments (tests using cold allodynia, acetone solution) were performed at the day intervals of 1, 2, 3, 5, 7, 10 and 14 after the completion of surgery. The higher the score, the more the animal models repeat the act of kicking or licking legs, which means the degree of pain is severe. The highest score was point 9. In the case of no response, the lowest score, 0, was applied. The act of moving and kicking legs may score point 1, and the act of continuously crouching or repeatedly kicking legs may score point 2. When the animal models repeated the act of licking and kicking their legs, the score was point 3. Each mouse was repeatedly tested three times and the sum score was measured.

FIG. 7 shows images from which the pain reduction effect of PLGA-CD-DEX-RVC according to an embodiment was confirmed:

FIG. 7A shows a schematic diagram showing an animal experiment using a chronic constriction injury (CCI) animal model and the effect of PLGA-CD-DEX-RVC; and FIG. 7B shows a graph showing the pain reduction effect of PLGA-CD-DEX-RVC obtained through pain evaluation (cold allodynia).

As a result, as shown in FIG. 7B, it was confirmed that the pain was the most reduced in the group treated with PLGA-CD-DEX-RVC. These results indicate that DEX-RVC can be effectively used for neurogenic pain.

### Experimental Example 7. Confirmation of In Vivo Degradation

It was confirmed whether the PLGA-CD-DEX-RVC nanofibers prepared in Example 1 were degraded in vivo.

Specifically, in order to measure the time during which PLGA melts in vivo, CY 5.5 fluorescent dye was attached to PLGA and PLGA-CD-DEX-RVC nanofibers and the results were implanted in the back integument of mice. Pearl Impulse small animal imaging carrier (LI-COR Biosciences, Lincoln, Nebr.) equipment was used, and the degree of degradation of nanofibers was measured by measuring the degree of reduced fluorescence expression.

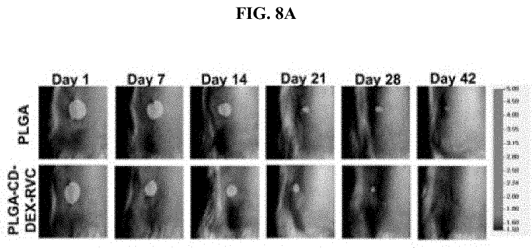

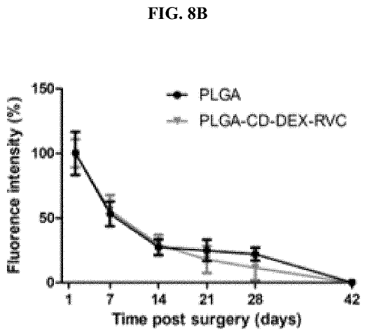

FIG. 8 shows an in vivo decomposition effect of PLGA-CD-DEX-RVC according to an embodiment:

FIG. 8A shows images showing the degradation of PLGA-CD-DEX-RVC in vivo over time; and FIG. 8B shows a graph showing the degradation of PLGA-CD-DEX-RVC in vivo over time.

As a result, as shown in FIGS. 8A and 8B, like PLGA, which is a biodegradable polymer, PLGA-CD-DEX-RVC was also well degraded in vivo. These results indicate that even when PLGA-CD-DEX-RVC is injected into a living body and used, it does not remain in the living body but is degraded and is stable.

### Experimental Example 8. Confirmation of Pain Marker Expression

It was confirmed whether the PLGA-CD-DEX-RVC nanofibers prepared in Example 1 reduced the expression of transient receptor potential vanilloid 1 (TRPV1) marker, which is known as a nociceptor, and the expression of Iba1 (ionized calcium binding adaptor molecule 1) marker for microglia, which is an inflammatory cell.

Neuropathic pain occurs due to a signal transduction of damaged nerve cells or an increase in inflammatory response in sensory neurons. Pain signals are transmitted from sensory neurons in the dorsal root ganglia (DRG) to the dorsal horn of the spinal cord.

Specifically, as in Experimental Example 6, the mice were perfused two weeks after the surgery to extract the spinal cord and DRG, and then immobilized with 4% PFA. After paraffin embed, the cells were cut to a size of 5 μm, and attached to a slide, followed by immunohistofluorescence staining. The cells were stained with an TRPV1 marker and an NeuN marker, which were used as antibodies for staining nuclei of neurons. Mouse anti-TRPV1 and rabbit anti-NeuN were used as primary antibodies, and Alexa 488 or Alexa 568 (Molecular Probes), and streptavidin-Alexa 594 were used as secondary antibodies. After staining, the cells were mounted and images thereof were captured by Confocal, and the expression of TRPV1, a pain marker, in neurons was quantified.

In addition, neurogenic pain is often increased depending on the inflammatory response. Accordingly, microglia, which is an inflammatory cell appearing in the central nervous carrier, was confirmed by paraffin-sectioning through Iba1 marker as described above and immunohistofluorescence staining. Goat anti-iba1 (1:500) was used as the primary antibody, and donkey anti goat 647 (1:1000, Invitrogen) was used as the secondary antibody. After staining, the cells were mounted and images thereof were captured by Confocal, and the expression of Iba1 in neurons was quantified.

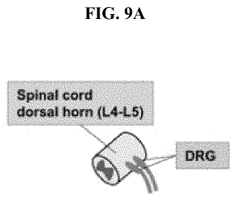

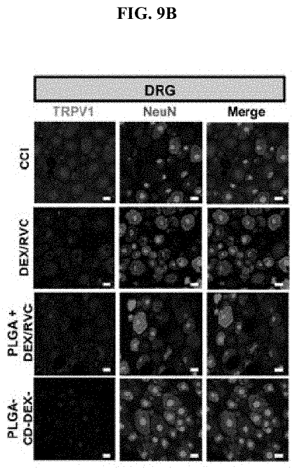

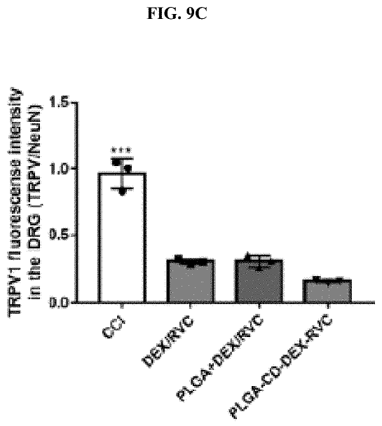

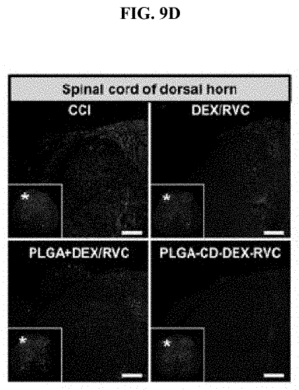

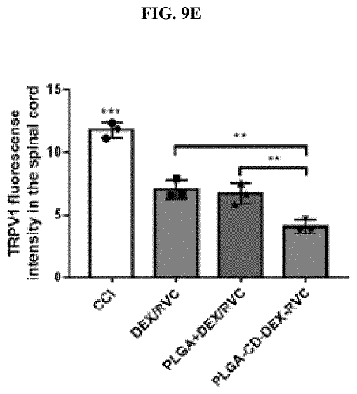

FIG. 9 shows the expression levels of TRPV1, a pain factor, when PLGA-CD-DEX-RVC according to an embodiment was used for treatment after nerve injury, wherein the expression levels were identified by immunofluorescence staining:

FIG. 9A shows a diagram illustrating that a pain signal is transferred from sensory neurons of the dorsal root ganglia (DRG) to the dorsal horn of spinal cord; FIG. 9B shows an image showing the expression of TRPV1, a pain marker, in DRG (Scale bar: 20 μm); FIG. 9C shows a graph of the quantified expression of TRPV1, a pain marker, in DRG; FIG. 9D shows an image showing the expression of TRPV1, a pain marker, in the spinal cord of dorsal horn (Scale bar: 100 μm); and FIG. 9E shows a graph of the quantified expression of TRPV1, a pain marker, in the spinal cord of dorsal horn.

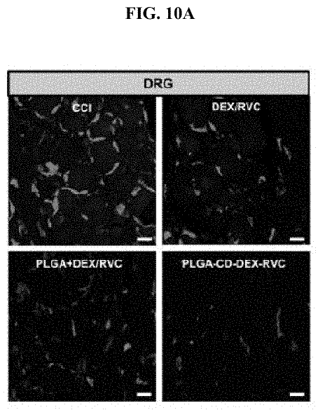

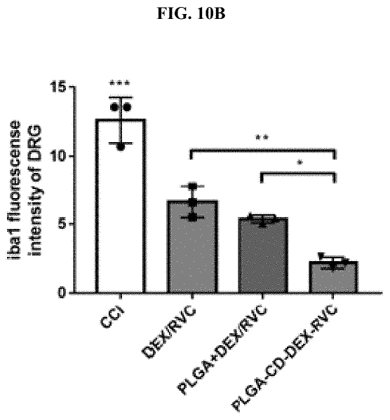

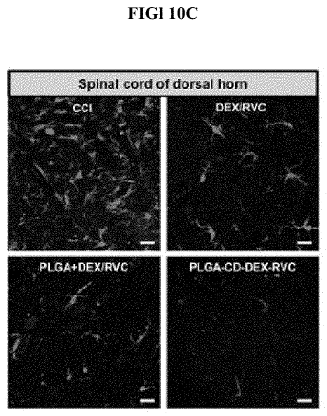

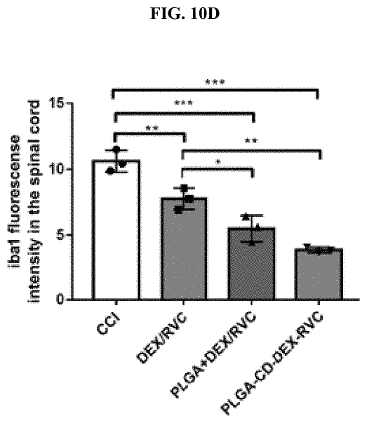

FIG. 10 shows the expression levels of Iba1, an inflammatory factor, when PLGA-CD-DEX-RVC according to an embodiment was used for treatment after nerve injury, wherein the expression levels were identified by immunofluorescence staining:

FIG. 10A shows an image showing the expression of Iba1, an inflammatory marker, in DRG (Scale bar: 20 μm); FIG. 10B shows a graph of the quantified expression of Iba1, an inflammatory marker, in DRG; FIG. 10C shows an image showing the expression of Iba1, an inflammatory marker, in the spinal cord of dorsal horn (Scale bar: 20 μm); and FIG. 10D shows a graph of the quantified expression of Iba1, an inflammatory marker, in the spinal cord of dorsal horn.

As a result, as shown in FIG. 9, it was confirmed that the expression of TRPV1 was the most reduced in the group treated with PLGA-CD-DEX-RVC. In addition, as shown in FIG. 10, the expression of Iba1 decreased the most in the group treated with PLGA-CD-DEX-RVC, confirming that microglia were significantly reduced compared to other groups. These results indicate that PLGA-CD-DEX-RVC is very effective in reducing pain.

When the drug delivery carrier according to an aspect is used, the time during which a drug stays in the living body may be prolonged, and due to the biodegradation thereof, few side effects occur.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A drug delivery carrier comprising:
polylactic-co-glycolic acid (PLGA) and β-cyclodextrin containing a drug,
wherein PLGA and the β-cyclodextrin are linked to each other by a linker.

2. The drug delivery carrier of claim 1, wherein PLGA and the β-cyclodextrin each have a thiol group.

3. The drug delivery carrier of claim 1, wherein the linker is a disulfide bond, and the thiol group of PLGA and the thiol group of the β-cyclodextrin form a disulfide bond.

4. The drug delivery carrier of claim 1, wherein a ratio of a glycolic acid to a lactic acid in PLGA is 3:1.

5. The drug delivery carrier of claim 1, wherein the drug delivery carrier has a porosity of about 30 vol % to about 50 vol %.

6. The drug delivery carrier of claim 1, wherein the drug is a pain treatment agent or an anesthetic agent.

7. The drug delivery carrier of claim 6, wherein the pain treatment agent is selected from the group consisting of celecoxib, diclofenac, diflunisal, piroxicam, meloxicam, etodolac, mefenamic acid, meclofenamic acid, ibuprofen, indometacin, ketoprofen, ketorolac, nabumetone, naproxen, nimesulide, sulindac, tepoxalin, tolmetin, neostigmine, magnesium, atropine, dexamethasone, prednisolone, prednisone, methyl prednisolone, triamcinolone, hydrocortisone, deflazacourt, betamethasone, budenoside, ketorolac, octreotide, ziconitide, droperidol, methotrexate, and haloperidol.

8. The drug delivery carrier of claim 6, wherein the anesthetic agent is selected from the group consisting of bupivacaine, levobupivacaine, ropivacaine, prilocaine, mepivacaine, benzocaine, tetracaine, and lidocaine.

9. The drug delivery carrier of claim 1, wherein the drug is released in a sustained manner.

10. The drug delivery carrier of claim 1, wherein the drug delivery carrier is produced by linking the β-cyclodextrin containing the drug to PLGA.

11. The drug delivery carrier of claim 1, wherein PLGA is electrospun to form nanofibers after a thiol end group is formed.

12. A method of preventing or treating a pain disorder comprising:
administering, to a subject in need thereof, a composition including PLGA and 3-cyclodextrin containing a pain treatment agent, wherein PLGA and the β-cyclodextrin are linked to each other by a linker.

13. The method of claim 12, wherein PLGA and the β-cyclodextrin each have a thiol group.

14. The method of claim 12, wherein the linker is a disulfide bond, and the thiol group of PLGA and the thiol group of the β-cyclodextrin form a disulfide bond.

15. The method of claim 12, wherein the β-cyclodextrin further contains an anesthetic agent.

16. The method of claim 12, wherein the pain disorder is caused by one selected from the group consisting of neuropathic pain, osteoarthritis, rheumatoid arthritis, fibromyalgia, back and musculoskeletal pain, spondylitis, intervertebral disk escape, spinal canal stenosis, juvenile rheumatoid arthritis, diabetic neuropathy, spontaneous pain, hypersensitivity pain, phantom limb pain, complex regional pain syndrome migraine, toothache, abdominal pain, ischemic pain, and post-operative pain.

17. A method of preparing a drug delivery carrier, the method comprising
forming a thiol end group in polylactic-co-glycolic acid (PLGA);
entrapping a drug in β-cyclodextrin having a thiol group; and
linking PLGA and the β-cyclodextrin via a disulfide bond.

18. The method of claim 17, further comprising electrospinning PLGA having the thiol end group therein.
